(19) United States
(12) Patent Application Publication
(10) Pub. No.: US 2020/0066382 A1
(43) Pub. Date: Feb. 27, 2020

(54) Patient Data Exchange (PDX-TEC)

(71) Applicant: Debra L. Griffin, Jackson, MS (US)

(72) Inventor: Debra L. Griffin, Jackson, MS (US)

(21) Appl. No.: 16/672,422

(22) Filed: Nov. 1, 2019

Related U.S. Application Data
(62) Division of application No. 14/573,392, filed on Dec. 17, 2014, now abandoned.
(60) Provisional application No. 61/999,310, filed on Jul. 23, 2014.

(51) Int. Cl. G16H 10/60; G16H 15/00

(52) U.S. Cl. CPC G16H 10/60; G16H 15/00

(57) ABSTRACT

Ppx-tec serves medical patients and providers by providing a quick and secure bluetooth connection to transfer medical results, insurance payment status between the patient and doctor during an appointment's entry and exit, providing for a comprehensive health record for user and family. No existing network required, the connection is temporary and only exists and operates for the transfer of this single packet of data. No Internet connection needed.

### CROSS-REFERENCE TO RELATED APPLICATIONS

This application is a divisional application claiming the benefit of Non-Provisional patent application Ser. No. 14/573,392, filed on Dec. 17, 2014, pursuant to 35 U.S.C. §§ 120 and 121. This application and Non-Provisional patent application Ser. No. 14/573,392 further claim the benefit of Provisional Patent Application No. 61/999,310, filed on Jul. 23, 2014, pursuant to 35 U.S.C. § 119(e).

### BRIEF SUMMARY OF THE INVENTION

Pdx-tec is a secure Bluetooth personal health records application that provides a platform for electronic health records (“EHRs”) by pairing doctors with patients as application users. The secure transmission of health data over Bluetooth is quick, simple and allows data to be both sent and received. Pdx-tec's patient users exchange data with pdx-tec's provider users within the application, which allows for EHR platform integration.

### BRIEF DESCRIPTION OF THE DRAWINGS

FIG. 1 is a block diagram illustrating the user interface of an application according to the present invention and the flow of data to other components of a health data system according to the present invention.

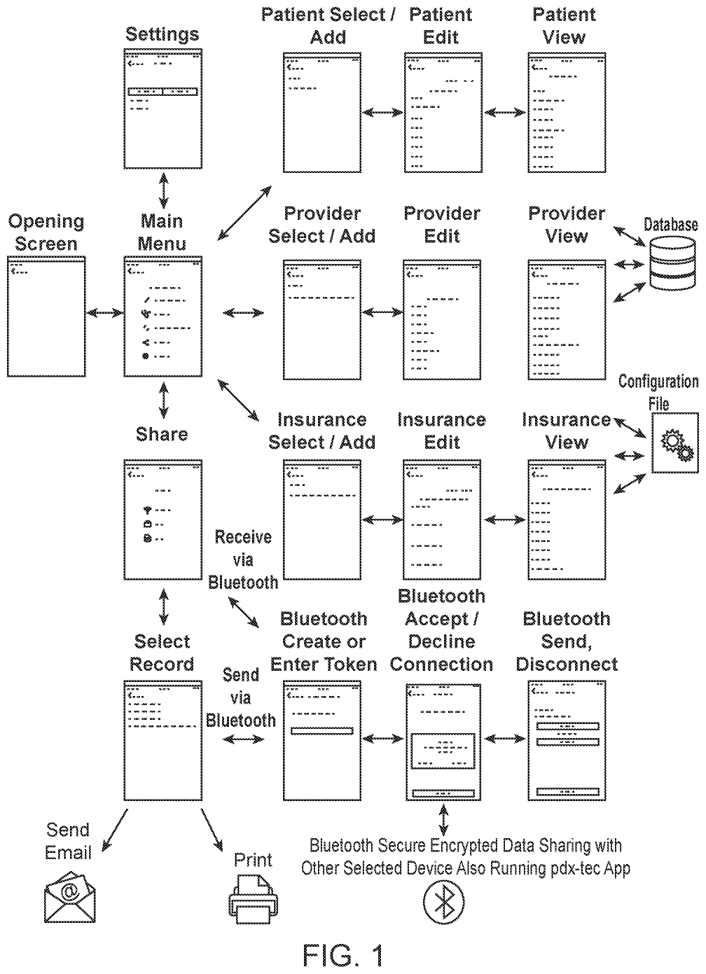

FIG. 2 is a block diagram illustrating an example sequence of data transfers according to the present invention.

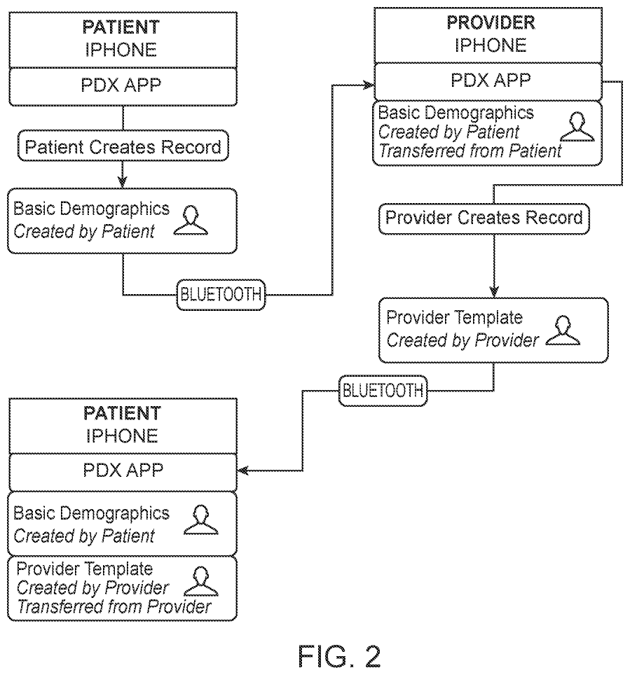

FIG. 3 is a block diagram illustrating an alternative example sequence of data transfers according to the present invention.

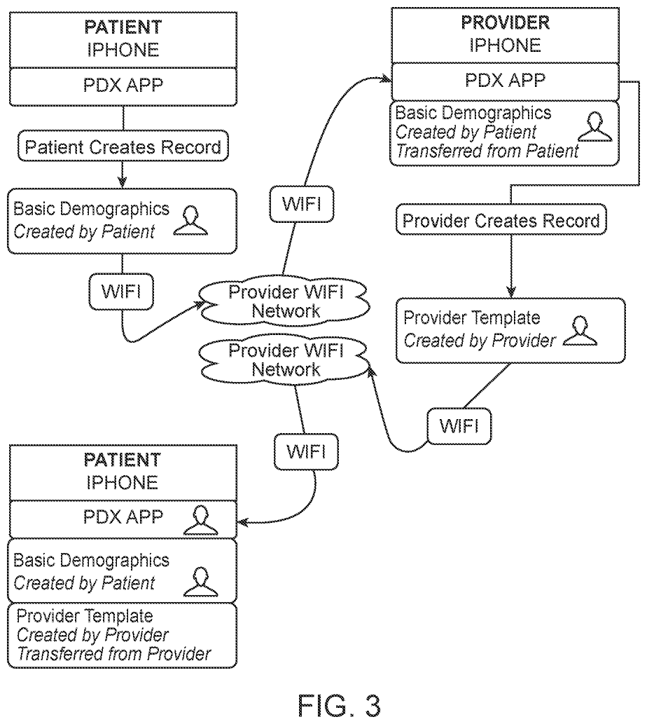

FIG. 4 is a block diagram illustrating an alternative example sequence of data transfers according to the present invention.

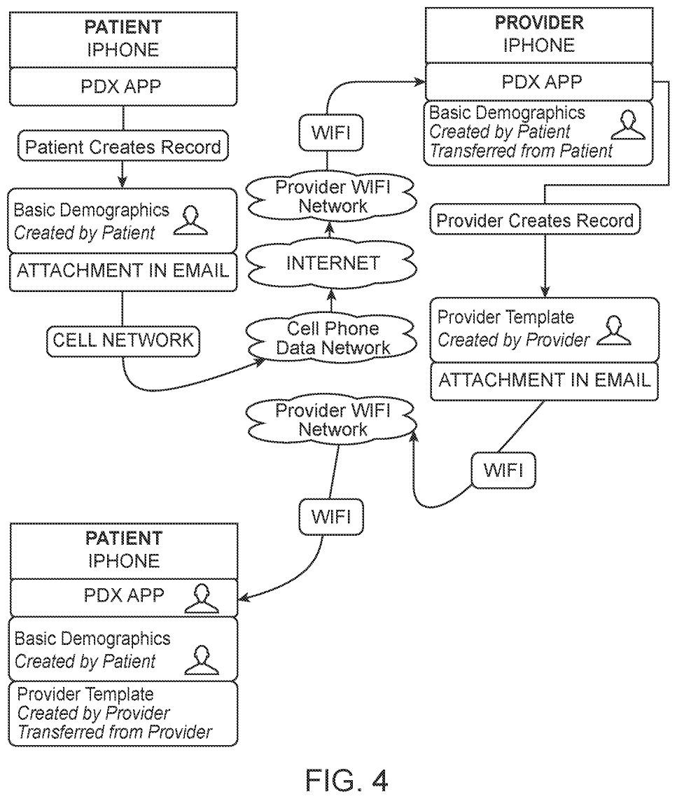

FIG. 5 is a block diagram illustrating an alternative example sequence of data transfers according to the present invention.

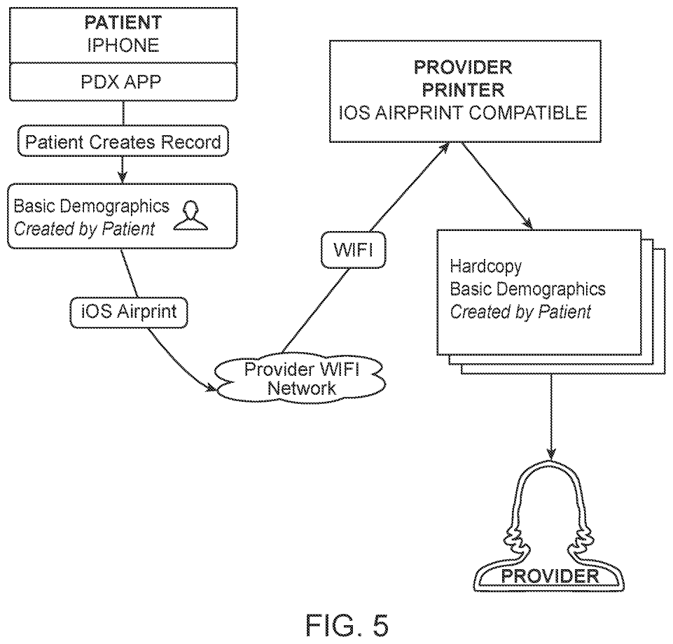

### DETAILED DESCRIPTION OF THE INVENTION

Pdx-tec serves medical patients and providers by providing a quick and secure Bluetooth method to transfer medical results between the two during an appointment's entry and exit for creating a comprehensive health record for both user and family.

The following features illustrate the convenience and other advantages of pdx-tec:

(a) Patients fill in and update information once, instead of at every new medical visit—which can be time consuming depending on the patient's medical history.

(b) When physically at a provider's office, the patient user can send and receive health results to/from staff by connecting with paired pdx-tec's apps.

(c) The provider's office receives the patient data and can return results to the patient.

(d) No internet connection required, but the patient and the provider users must be in physical proximity.

(e) Data is secure; no one else sees the data, even if they also have the app.

(f) Paired with an 8-character token assigned by provider, Bluetooth transmission is point-to-point. A token is another layer of security for allowing devices to talk and is uniquely different on every transmission.

(g) The connection is temporary—only for the duration of the visit—and can be disconnected at any time by the patient or the provider.

(h) No additional cost in using Bluetooth for the patient or provider, if they already have a Bluetooth-enabled mobile device.

(i) Can include built-in insurance information tracker component.

The invention claimed is:

1. A method of exchanging personal health records between a first mobile device and a second mobile device, comprising:
a) collecting health data, including information regarding one or more of the health, diagnosis, treatment, or health insurance of a patient;
b) creating a personal health record by storing the health data in a database located on the first mobile device;
c) establishing a point-to-point wireless connection between the first mobile device and the second mobile device;
d) selecting and retrieving the personal health record from the database stored on the first mobile device;
d) encrypting the personal health record using the first mobile device;
e) transmitting the encrypted personal health record from the first mobile device to the second mobile device over the point-to-point wireless connection;
f) receiving and decrypting the encrypted personal health record using the second mobile device; and
g) storing the personal health record in a database located on the second mobile device.

2. The method of exchanging personal health records of claim 1, wherein the point-to-point wireless connection between the first mobile device and the second mobile device is established according to the Bluetooth standard.

3. The method of exchanging personal health records of claim 1, wherein the encryption and decryption of the personal health record is performed by using a Bluetooth token.

4. The method of exchanging personal health records of claim 1, wherein the collection of health data step includes establishing a network connection between the first mobile device and an electronic health record system and downloading health data, including information regarding one or more of the health, diagnosis, treatment, or health insurance of a patient, from the electronic health record system to the first mobile device over the network connection.

5. The method of exchanging personal health records of claim 1, further comprising the steps of:
h) establishing a network connection between the second mobile device and an electronic health record system;
i) selecting and retrieving the personal health record from the database stored on the second mobile device; and
j) uploading the personal health record from the second mobile device to the electronic health record system using the network connection.
